(19) United States
(12) Patent Application Publication
(10) Pub. No.: US 2022/0040379 A1
(43) Pub. Date: Feb. 10, 2022

(54) NEW USE OF STEM CELL GENERATOR IN PREPARATION OF BONE DEFECT REPAIR MATERIALS

(71) Applicant: EAST CHINA UNIVERSITY OF SCIENCE AND TECHNOLOGY, Shanghai (CN)

(72) Inventors: Changsheng LIU, Shanghai (CN); Kai DAI, Shanghai (CN); Jing WANG, Shanghai (CN); Shunshu DENG, Shanghai (CN)

(21) Appl. No.: 17/427,720

(22) PCT Filed: Jan. 21, 2020
(86) PCT No.: PCT/CN2020/073592

§ 371 Date: Aug. 2, 2021

(30) Foreign Application Priority Data
Jan. 31, 2019 (CN) 201910100503.9

(51) Int. Cl. A61L 27/38; A61L 27/54; A61L 27/36; A61L 27/22

(52) U.S. Cl. CPC A61L 27/3834; A61L 27/54; A61L 27/365; A61L 2430/02; A61L 2300/252; A61L 2300/412; A61L 2300/414; A61L 27/222

(57) ABSTRACT

Disclosed is a new use of a stem cell generator in preparation of bone defect repair materials, wherein the stem cell generator is formed by implanting a biomaterial with osteogenic induction capability or a biomaterial loaded with active substances and/or cells into an animal or a human body and generating organoids after development, the active substances are bone morphogenetic protein-2, or bone morphogenetic protein-7, other growth factors/polypeptides having bone regeneration induction ability, growth factors/polypeptide combinations, or a combination thereof. The cells are bone marrow-derived mesenchymal stem cells, adipose-derived mesenchymal stem cells or other derived mesenchymal stem cells; other types of cells with osteogenic differentiation capability; cells that aid in osteogenic differentiation of mesenchymal stem cells, such as vascular endothelial cells and the like. The stem cell generator is used to prepare bone repair materials for treatment of various types of bone defects or bone deformities that are spontaneous or caused by trauma.

### TECHNICAL FIELD

The invention relates to the crossing filed of material, life and medicine, and relates to a novel application method for bone-like organs formed by stem cell generators in vivo. The bone-like organs produced by stem cell generators in vivo can be used to treat spontaneous or trauma-induced bone defect or deformity.

### BACKGROUND

As the main mechanics bearing system of the human body, bone determines the human's athletic ability. Meanwhile, bone as important endocrine organ is involved in regulating many physiological processes. Damage to the bones will seriously affect the quality of life of the individual. Although a variety of artificial bone products have been developed, most of the artificial bone products used in clinic is still insufficient in activity, which is difficult to meet the clinical treatment requirement of large-scale bone defects caused by disease or trauma. Even more serious is that with the coming of an aging society, the incidence of bone injury continues to rise. For the treatment of this type of bone defect, autologous bone graft as the gold standard can achieve good therapeutic effects. However, the area and quantity of autologous bone are limited, and autologous bone removal can cause persistent pain in the donor site. The treatment effect of secondary fracture or bone defect is not good. In addition, other spontaneous diseases, such as different length of limbs, maxillofacial bone loss, and femoral head necrosis, also require bone transplantation.

In order to deal with the disadvantages of autologous bone transplantation, various organic and inorganic biomaterials have been developed for bone defect treatment. However, most of the biomaterials generally do not have or only have very low biological activity, and the treatment effect of large-scale bone defects or ischemic osteonecrosis is not good, especially in the treatment of elderly patients. However, allogeneic bone, as another autologous bone substitute with better therapeutic effect, has the possibility of pathogen contamination and immunogenicity.

### SUMMARY OF THE INVENTION

The object of the present invention is to provide a new method for treating bone defects caused by various reasons by using bone-like organs produced by the constructed stem cell generator.

The first aspect of the present invention provides a stem cell generator, which is formed by implanting a biomaterial with osteoinductive ability or a biomaterial loaded with an active substance and/or cell into an animal or human body to develop and generate an organoid, wherein the active substance is bone morphogenetic protein-2 (BMP-2), bone morphogenetic protein-7 (BMP-7), other growth factor/polypeptide having the ability to induce bone regeneration, growth factor/polypeptide combination, or combination thereof; the cell is mesenchymal stem cell, and the mesenchymal stem cell is bone marrow-derived mesenchymal stem cell, adipose-derived mesenchymal stem cell, or mesenchymal stem cell from other sources; other type of cell having osteogenic differentiation ability; a cell assisting mesenchymal stem cell in osteogenic differentiation, such as vascular endothelial cell and the like.

In another preferred example, the biomaterial is selected from one of collagen, gelatin, chitosan, alginic acid, hyaluronic acid, bacterial cellulose, polylactic acid, polyglycolide, polylactide, polyhydroxy fatty acid ester, polycarbonate, polycaprolactone, polyethylene glycol, polyfumaric acid, hydroxyapatite, calcium sulfate, tricalcium phosphate, tetracalcium phosphate, octacalcium phosphate, calcium metaphosphate, magnesium phosphate, pyrophosphate, calcium silicate, bioglass and decalcified bone matrix, or a copolymer/blend composition thereof.

In another preferred example, the biomaterial is autologous bone or allogeneic bone.

In another preferred example, the organoid contains pluripotent stem cells and bone marrow cells.

In another preferred embodiment, the pluripotent stem cell is hematopoietic stem/progenitor cell (HSC/HPC), mesenchymal stem cells (MSC) or other type of pluripotent stem cell.

In another preferred example, the animal or human body refers to the muscle pocket, muscle space, intra-muscle, subcutis, or dorsal muscle of the abdominal cavity of the animal or human.

In another preferred example, the mass ratio of the active substance to the biomaterial is 0.0001-1:1.

In another preferred example, the number of cells inoculated is 1×10⁵-5×10⁸ cells per 100-150 mm³ of biomaterial.

In vivo stem cell generator is bone-like organ formed by developing a biomaterial loaded with an active substance and/or cell, or a biomaterial with osteoinductive ability in vivo. The stem cell generator can grow and develop in the body to form a tissue with bone-like organ having a microscopic bone structure and vascularization characteristics similar to normal bone.

The research results of the present invention show that the bone-like organ produced by the in vivo stem cell generator can repair critical-sized bone defect, and is expected to be applied to the clinical treatment of severe bone defects, bone nonunion, and elderly patients with weak regenerative ability.

The second aspect of the present invention provides the method for constructing the stem cell generator according to the first aspect, comprising the following steps:

(1) implanting a biomaterial into an animal or human body;

(2) generating an organoid after development in the body to form the stem cell generator, wherein,

the biomaterial is a biomaterial loaded with an active substance and/or cell, or a biomaterial having osteoinductive ability.

In another preferred example, the active substance is bone morphogenetic protein-2 (BMP-2), bone morphogenetic protein-7 (BMP-7), osteogenic peptide, other growth factor or polypeptide having the ability to induce bone regeneration and angiogenesis, such as VEGF, PDG, or a combination of the growth factor/polypeptide.

In another preferred example, the bone morphogenetic protein-2 is recombinant bone morphogenetic protein-2.

In another preferred example, the bone morphogenetic protein-7 is recombinant bone morphogenetic protein-7.

In another preferred example, the biomaterial is selected from one of collagen, gelatin, chitosan, alginic acid, hyaluronic acid, bacterial cellulose, polylactic acid, polyglycolide, polylactide, polyhydroxy fatty acid ester, polycarbonate, polycaprolactone, polyethylene glycol, polyfumaric acid, hydroxyapatite, calcium sulfate, tricalcium phosphate, tetracalcium phosphate, octacalcium phosphate, calcium metaphosphate, magnesium phosphate, pyrophosphate, calcium silicate, bioglass and decalcified bone matrix, or a copolymer/blend composition thereof.

In another preferred example, the mass ratio of the active substance to the biomaterial is 0.0001-1:1.

In another preferred example, the cell is mesenchymal stem cell, and the mesenchymal stem cell is bone marrow-derived mesenchymal stem cell, adipose-derived mesenchymal stem cell, or mesenchymal stem cell from other sources; other type of cell having osteogenic differentiation ability; a cell assisting mesenchymal stem cell in osteogenic differentiation, such as vascular endothelial cell and the like.

In another preferred example, the number of cells inoculated is 1×10⁵-5×10⁸ cells per 100-150 mm³ of biomaterial.

In another preferred example, the animal or human body refers to the muscle pocket, muscle space, intra-muscle, subcutis, or dorsal muscle of the abdominal cavity of the animal or human.

In the present invention, the organoid has structures and functions similar to those of native bone, including complete bone tissue, bone marrow-like tissue and various functional stem cells.

In another preferred example, the organoid contains stem cell, and the stem cell is hematopoietic stem/progenitor cell, mesenchymal stem cell, endothelial progenitor cell or other types of pluripotent stem cell.

The third aspect of the present invention provides a method for preparing a bone graft/filler, the method comprising the following steps:

(1) implanting a biomaterial into an animal or human body;

(2) generating an organoid after development in the body to obtain the bone graft/filler, wherein,

the biomaterial is a biomaterial loaded with bone morphogenetic protein-2, or bone morphogenetic protein-7, or other growth factor/polypeptide capable of inducing bone regeneration or a combination of the growth factor/polypeptide.

In another preferred example, the biomaterial is selected from one of collagen, gelatin, chitosan, alginic acid, hyaluronic acid, bacterial cellulose, polylactic acid, polyglycolide, polylactide, polyhydroxy fatty acid ester, polycarbonate, polycaprolactone, polyethylene glycol, polyfumaric acid, hydroxyapatite, calcium sulfate, tricalcium phosphate, tetracalcium phosphate, octacalcium phosphate, calcium metaphosphate, magnesium phosphate, pyrophosphate, calcium silicate, bioglass and decalcified bone matrix, or a copolymer/blend composition thereof.

In another preferred example, the mass ratio of the active substance to the biomaterial is 0.0001-1:1.

In another preferred example, the animal or human body refers to the muscle pocket, muscle space, intra-muscle, subcutis, or dorsal muscle of the abdominal cavity of the animal or human.

The fourth aspect of the present invention provides use of the stem cell generator according to the first aspect for manufacturing a bone repair material or as a bone repair material.

In another preferred example, the bone repair material is used to treat spontaneous or trauma-induced bone defect or bone deformity.

In another preferred example, the method for repairing bone defect is used in the following occasions or disease treatment:

(1) for bone graft treatment of bone injury caused by trauma, bone nonunion, and delayed bone healing;

(2) for the treatment of spinal fusion and bone defect caused by bone tumor, osteoporosis, bone deformity and other diseases;

(3) for the treatment of bone defect in elderly patients with weak regenerative ability;

(4) for the treatment of other diseases that require bone transplantation.

The fifth aspect of the present invention provides a method for repairing bone defect, wherein a bone-like organ produced by a stem cell generator is used to replace autologous bone and/or other biomaterials for bone defect repair.

In another preferred example, a method for repairing critical-sized bone defect is provided, wherein a bone-like organ produced by an in vivo stem cell generator is used to replace autologous bone and/or other biomaterials for bone defect repair.

In another preferred example, the bone-like organ used for bone repair is derived from a biomaterial loaded with a growth factor and/or cell, or a biomaterial with osteoinductive ability, which is implanted into animal/human muscle pocket or subcutaneous part, etc. to constitute a stem cell generator and form a bone-like organ by developing over a period of time, in which the mass ratio of the active substance to the biomaterial is 0.0001-1:1, and the number of cells used for inoculation is 1×10⁵-5×10⁸.

In another preferred example, the growth factor used is bone morphogenetic protein-2, bone morphogenetic protein-7, or other growth factor/polypeptide capable of inducing bone regeneration or a combination of the growth factor/polypeptide.

In another preferred example, the cell is adipose-derived mesenchymal stem cell, bone marrow-derived mesenchymal stem cell, other type of cell having osteogenic differentiation ability, or a combination thereof.

In another preferred example, the biomaterial is collagen, gelatin, chitosan, alginic acid, hyaluronic acid, bacterial cellulose, polylactic acid, polyglycolide, polylactide, polyhydroxy fatty acid ester, polycarbonate, polycaprolactone, polyethylene glycol, polyfumaric acid, hydroxyapatite, calcium sulfate, tricalcium phosphate, tetracalcium phosphate, octacalcium phosphate, calcium metaphosphate, magnesium phosphate, pyrophosphate, calcium silicate, bioglass and decalcified bone matrix with good biocompatibility, or a copolymer/blend composition thereof.

In another preferred example, the resulting bone-like organ has a structure and function similar to that of autologous bone.

In another preferred example, the bone-like organ used for bone repair is a new tissue induced by a stem cell generator in the body.

In another preferred example, the bone defects are various spontaneous or trauma-induced bone defects or bone deformities.

In another preferred example, the method for repairing bone defect is used in the following occasions or disease treatment:

(1) for bone graft treatment of bone injury caused by trauma, bone nonunion, and delayed bone healing;

(2) for the treatment of spinal fusion and bone defect caused by bone tumor, osteoporosis, bone deformity and other diseases;

(3) for the treatment of bone defect in elderly patients with weak regenerative ability;

(4) for the treatment of other diseases that require bone transplantation.

In another preferred example, the disease treatment includes the following diseases or conditions:

(1) bone defect/loss caused by trauma or disease;

(2) hip-preserving treatment for early ischemic femoral head necrosis;

(3) filling for osteoporosis, spinal compression fractures, etc.;

(4) treatment of other diseases that require bone grafting/filling.

The present invention proposes to use in vivo stem cell generator to construct a bone-like organ in ectopia by autologous development for bone defect treatment. The stem cell generator can provide a large-scale, functional, reproducible, and non-immunogenic bone-like organ.

Osteogenic active proteins represented by bone morphogenetic protein (BMP) have the effect of inducing ectopic bone formation, and with the assistance of biological materials, they induce the production of bone-like organ having a structure and function similar to autologous bone. The bone-like organ constructed by this method contains abundant blood vessel tissue, bone marrow tissue. Pathological sections also show that the resulting bone-like organs were similar in structure to autologous cortical and cancellous bone. In the present invention, a large-volume bone-like organ can be constructed in both young and old mice, and the critical-sized skull defect repair experiment shows that the constructed bone-like organ can quickly repair the critical-sized skull defect and has a good therapeutic effect. This method has the potential to replace traditional autologous bone graft, as an innovative treatment technique, it can be applied to the treatment of bone defects.

It should be understood that within the scope of the present invention, the above-mentioned each technical feature of the present invention and each technical feature specifically described thereafter (such as the examples) can be combined with each other to form a new or preferred technical solution. Each feature disclosed in the specification can be replaced by any alternative feature that provides the same, equal or similar purpose. Due to space limitations, they will not be repeated one by one.

### BRIEF DESCRIPTION OF THE FIGURES

FIG. 1 shows the overall experimental flow chart of the example.

FIG. 2 shows macroscopic views of bone-like organs produced by stem cell generators formed in young and old mice 3 weeks after the materials were implanted.

FIG. 3 shows the H&E stained sections of bone-like organs produced by stem cell generators formed in young and old mice 3 weeks after the materials were implanted.

FIG. 4 shows TRAP stained sections of bone-like organs produced by stem cell generators formed in young and old mice 3 weeks after the materials were implanted.

FIG. 5 shows CD31 immunofluorescence sections of bone-like organs produced by stem cell generators formed in young and old mice 3 weeks after the materials were implanted.

FIG. 6 shows typical flow cytometry diagrams of bone-like organs produced by stem cell generators formed in young and old mice 3 weeks after the materials were implanted.

FIG. 7 shows the flow cytometry statistics of bone-like organs produced by stem cell generators formed in young and old mice 3 weeks after the materials were implanted.

FIG. 8 shows the experimental process diagram of use of bone-like organs produced by stem cell generators developed in the body for three weeks in the repair of autologous skull defect in young mice.

FIG. 9 shows the μCT images after the bone-like organs produced by stem cell generators developed in the body for three weeks are used in the repair of autologous skull defect in young mice for 2 W, 4 W, and 6 W.

FIG. 10 shows the repair percentage statistics after the bone-like organs produced by stem cell generators developed in the body for three weeks are used in the repair of autologous skull defect in young mice for 2 W, 4 W, and 6 W.

FIG. 11 shows the BV/TV statistics after the bone-like organs produced by stem cell generators developed in the body for three weeks are used in the repair of autologous skull defect in young mice for 2 W, 4 W, and 6 W.

FIG. 12 shows the BMD statistics after the bone-like organs produced by stem cell generators developed in the body for three weeks are used in the repair of autologous skull defect in young mice for 2 W, 4 W, and 6 W.

FIG. 13 shows the H&E stained sections after the bone-like organs produced by stem cell generators developed in the body for three weeks are used in the repair of autologous skull defect in young mice for 2 W, 4 W, and 6 W.

FIG. 14 shows the TRAP stained sections after the bone-like organs produced by stem cell generators developed in the body for three weeks are used in the repair of autologous skull defect in young mice for 2 W, 4 W, and 6 W.

FIG. 15 shows the experimental process diagram of use of bone-like organs produced by stem cell generators developed in the body for three weeks in the repair of autologous skull defect in old mice.

FIG. 16 shows the μCT images after the bone-like organs produced by stem cell generators developed in the body for three weeks are used in the repair of autologous skull defect in old mice for 6 W.

FIG. 17 shows the repair percentage statistics after the bone-like organs produced by stem cell generators developed in the body for three weeks are used in the repair of autologous skull defect in old mice for 6 W.

FIG. 18 shows the BV/TV statistics after the bone-like organs produced by stem cell generators developed in the body for three weeks are used in the repair of autologous skull defect in old mice for 6 W.

FIG. 19 shows the BMD statistics after the bone-like organs produced by stem cell generators developed in the body for three weeks are used in the repair of autologous skull defect in old mice for 6 W.

FIG. 20 shows the H&E stained sections after the bone-like organs produced by stem cell generators developed in the body for three weeks are used in the repair of autologous skull defect in old mice for 6 W.

FIG. 21 shows the TRAP stained sections after the bone-like organs produced by stem cell generators developed in the body for three weeks are used in the repair of autologous skull defect in old mice for 6 W.

### DETAILED DESCRIPTION

After extensive and intensive researches, the inventors of the present application found that a biomaterial loaded with active substance or a biomaterial with activity can form stem cell generator in the body and develop into bone-like organ. This bone-like organ not only has cell components and tissue structure similar to autologous bone, but also has the function of bone tissue, and can be used as an effective substitute for bone graft/filler represented by autologous bone to treat bone defects.

The in vivo experimental study of the present invention shows that the bone-like organ developed by the stem cell generator formed after the material is loaded with BMP-2 has similar structure and function to autologous bone, and can replace autologous bone for bone repair. The pathological sections show that the bone marrow structure and bone structure of this bone-like organ and autologous bone were similar. Immunofluorescence staining and flow cytometry show that bone-like organ contains abundant blood vessels. The constructed stem cell generator can quickly repair critical-sized skull defects in young or old mice. This method provides a new way to obtain bone-like organ developed from autologous body. The resulting bone-like organ can effectively repair bone defects and is hoped to become a new source of clinical autologous bone transplantation to deal with the treatment of bone defect diseases with increasing incidence in the aging society.

The stem cell generator produced by the method of the present invention develops a bone-like organ with a structure and function similar to autologous bone, and can replace autologous bone for the repair or filling of various bone defects/losses.

In the present invention, a stem cell generator can be constructed by implanting active materials subcutaneously or in a muscle pocket, and the obtained stem cell generator can be used as a bone-like organ after trimming or other suitable operations and applied to the treatment of bone defect/loss and other orthopedic diseases.

In summary, based on the findings of the present invention, it is expected that the stem cell generator of the present invention can be developed into a bone-like organ for the treatment of various spontaneous or trauma-induced bone defects/losses and other orthopedic diseases.

Specifically, it can be applied to the following aspects:

1. various spontaneous or trauma-induced bone defects/losses;

2. hip-preserving treatment for early ischemic femoral head necrosis;

3. filling treatment of osteoporosis, spine compression fracture;

4. treatment of other related orthopedic diseases.

The present invention will be further described below in conjunction with specific examples. It should be understood that these examples are only used to illustrate the present invention and not to limit the scope of the present invention. The experimental methods without specific conditions in the following examples generally follow the conventional conditions (such as those described in Sambrook et al., Molecular Cloning: Laboratory Manual (New York: Cold Spring Harbor Laboratory Press, 1989) or the conditions recommended by the manufacturer.

Unless stated otherwise, percentages and parts are percentages by weight and parts by weight. Unless otherwise defined, all professional and scientific terms used herein have the same meaning as those familiar to the skilled in the art. In addition, any methods and materials similar to or equivalent to those described can be applied to the method of the present invention. The preferred implementation methods and materials described herein are for demonstration purposes only.

### Example 1 Preparation of Implant Material

30 μg of recombinant human bone morphogenetic protein-2 (rhBMP-2) synthesized by eukaryotic or prokaryotic expression system was added to a gelatin sponge (5 mm diameter=5 mm thick, 10 mg weight) and lyophilized to form an active material containing growth factor.

### Example 2 Bone-Like Organs Developed in Young Mice

The active materials described in Example 1 were implanted subcutaneously into the back of 8-week-old C57BL/6 male mice to form stem cell generators. After 3 weeks of feeding, the bone-like organs developed by the stem cell generator were taken out. One part was used to take macro photos, make H&E sections and flow cytometry detection, and the other part was used for the transplantation treatment of autologous skull defect.

### Example 3 Bone-Like Organs Developed in Old Mice

The active materials described in Example 1 were implanted subcutaneously into the back of 52-week-old C57BL/6 male mice to form stem cell generators. After 3 weeks of feeding, the bone-like organs developed by the stem cell generator were taken out. One part was used to take macro photos, make H&E sections and flow cytometry detection, and the other part was used for the transplantation treatment of autologous skull defect.

FIG. 1 showed the flow chart of the entire autologous skull defect transplantation treatment. The flow chart showed that the stem cell generators implanted in young/old mice developed into bone-like organs after 3 weeks, and one part were used for further characterization, the other part was used to treat autologous skull defects.

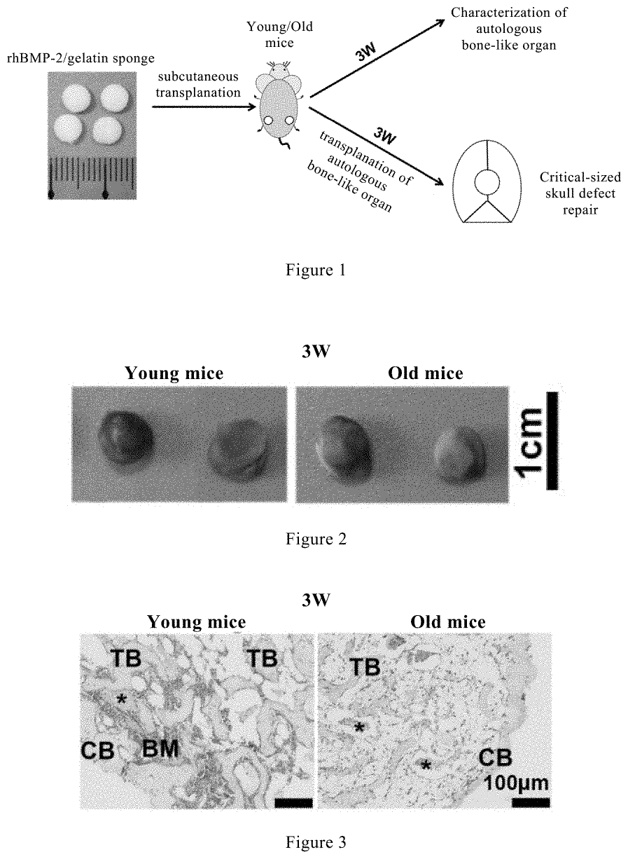

The macro photograph of FIG. 2 showed the stem cell generators formed in young/old mice in Example 2 and Example 3. The developed bone-like organs were dark red, indicating that they were rich in blood cells and blood vessel networks, and the tissue morphology thereof were also similar to autologous bone.

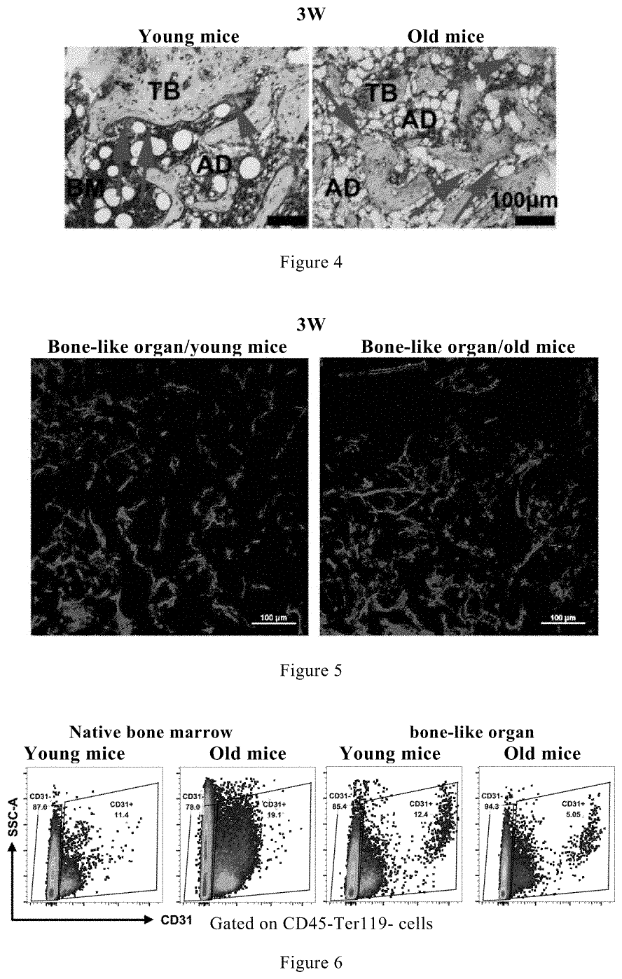

The H&E stained sections in FIG. 3 and the TRAP (tartrate-resistant acid phosphatase) stained sections in FIG. 4 together showed that the bone-like organs developed by the stem cell generators had microstructure and function similar to that of autologous bone.

The CD31 immunofluorescence staining shown in FIG. 5 proved that the bone-like organ developed by the stem cell generator had abundant vascular network. This bone-like organ was a highly vascularized bionic autologous bone, which could be used as an effective bone graft for the treatment of ischemic bone defects.

The flow cytometry detection results of FIG. 6 and FIG. 7 showed that the change trend of the proportion of CD31⁺ cells in bone-like organs constructed subcutaneously in mice of different ages was the same as that of the native bone marrow of mice of corresponding ages, namely, as the mice aged, the proportion of CD31⁺ cells therein showed a downward trend, but the proportion of CD31⁺ cells in old mice was significantly lower than that in young mice, suggesting that the blood vessel density in the native bone marrow in old mice was lower than that in young mice. This phenomenon was not found in bone-like organs, suggesting that the bone-like organs constructed in old mice had the characteristics of young bones.

### Example 4

Use of bone-like organs produced by stem cell generators in vivo for the treatment of autologous skull defects in young mice

The purpose of this example was to evaluate the therapeutic effect of the bone-like organ produced by the stem cell generator manufactured in the same young mouse on the 5 mm diameter defect of the young mouse's skull.

The active material used was the scaffold containing rhBMP-2 described in Example 1. The bone-like organs were produced by the development of stem cell generators in the animal body in Example 2.

Method:

SPF C57BL/6 mice, male, 8 weeks old, were randomly grouped. The experiment was grouped as follows.

| Group | Blank group | bone-like organ |
|---|---|---|
| Number | 6 | 6 |

Preparation of bone-like organ: the scaffold containing rhBMP-2 in Example 1 was subcutaneously implanted to produce bone-like organs after three weeks of development, and the bone-like organs were then removed and trimmed by using a punch with 5 mm inner diameter to obtain cylindrical bone-like organs with 5 mm diameter.

Autologous bone-like organ transplantation: After the mouse was anesthetized, the skin of the head of the mouse was cut open with a scalpel, and the skull was exposed. A circular saw with 5 mm outer diameter was used to create a 5 mm skull defect in the mouse, and the autologous bone-like organ prepared in the previous step was transplanted to the skull defect area. After the skin was sutured, the mouse was placed in a constant temperature stage to keep warm until the mice awoke. The samples were taken out for test at the established time point. The mice in the blank group only were made 5 mm skull defects, and then the wounds were sutured.

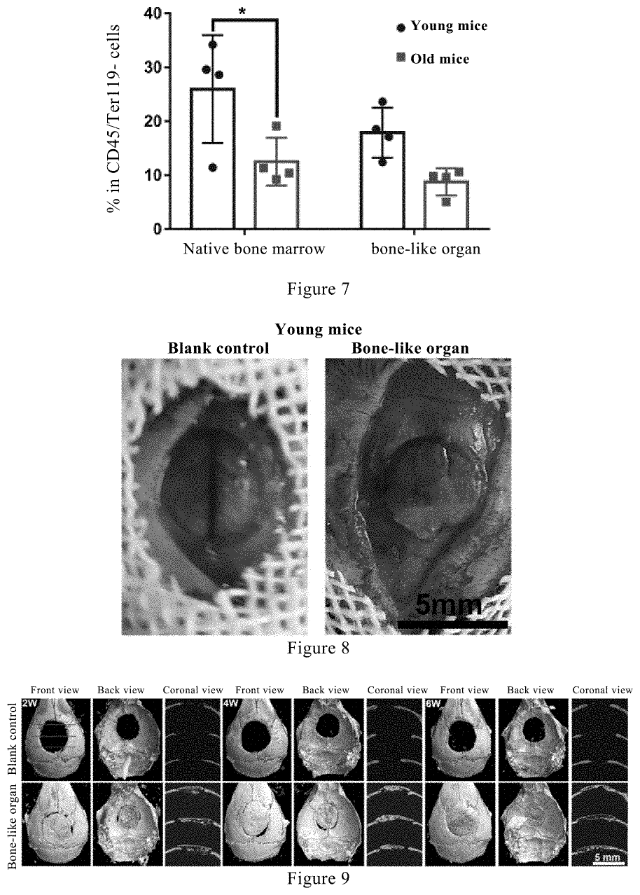

FIG. 8 showed an experimental process diagram of use of bone-like organs produced by the development of stem cell generators for treating autologous skull defects in young mice. The figure showed that after it was trimmed, the bone-like organ developed by the constructed stem cell generator in the body well covered the defect area and achieved the purpose of rapid repair.

FIG. 9 showed the μCT scan images of use of bone-like organs produced by the development of stem cell generators for treating autologous skull defects in young mice for 2 W, 4 W, and 6 W. The figure showed that the bone-like organs produced by the development of stem cell generators quickly repaired bone defects.

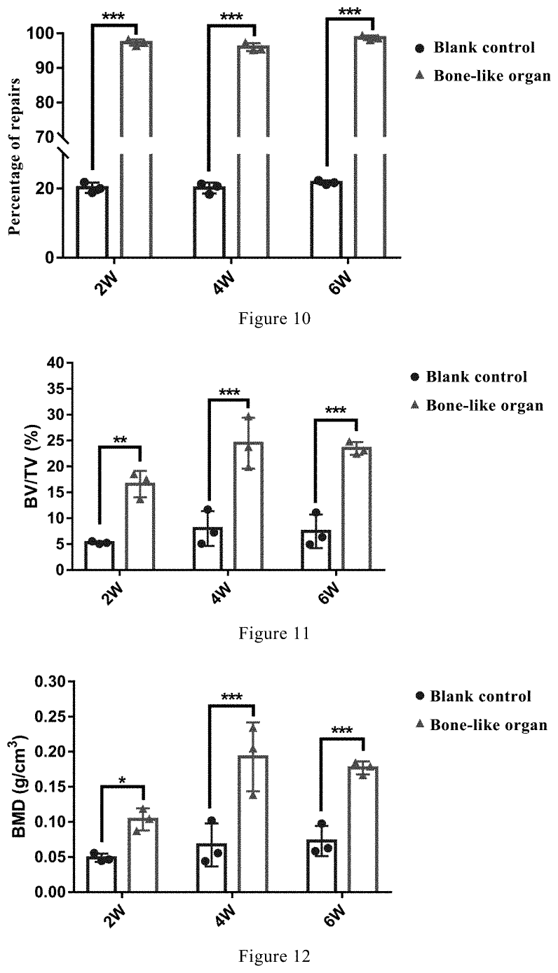

The quantitative data in FIG. 10 further showed that the bone-like organs produced by the development of stem cell generators achieved nearly 100% repair coverage of the bone defect.

FIGS. 11 and 12 showed that the BV/TV (bone volume/total volume) and BMD (bone mineralization density) of the repair site of bone-like organ produced by the development of stem cell generators were significantly higher than those of the blank control group, showing that bone-like organ produced by the development of stem cell generators had a better repair effect.

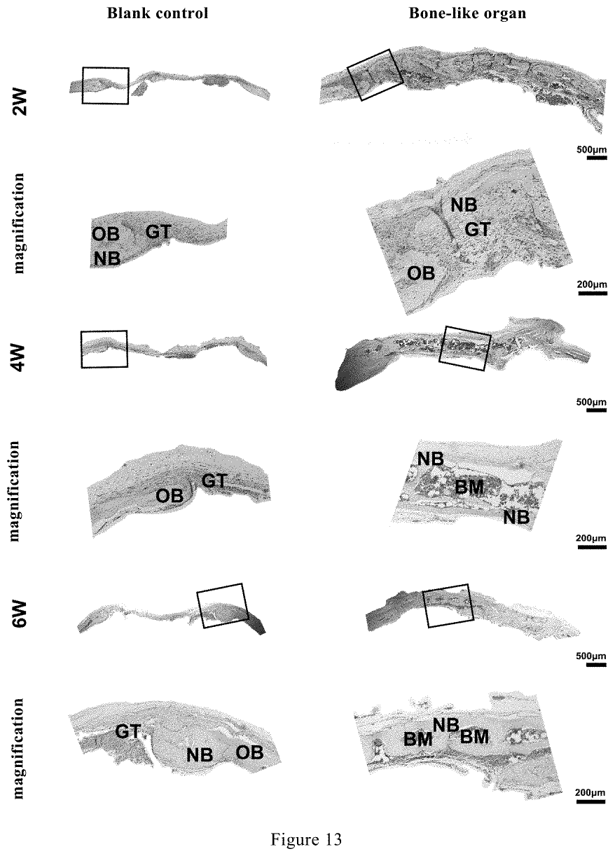

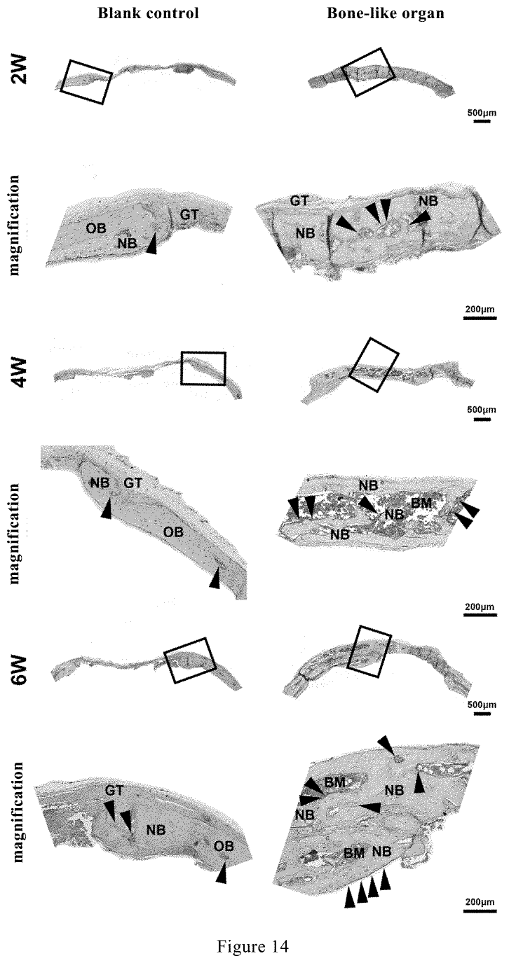

The H&E stained section images in FIG. 13 and the TRAP stained section images in FIG. 14 together indicated that the bone-like organ produced by the development of the stem cell generator could survive at the defect site after transplantation and effectively integrate with the defect edge to achieve a good repair effect.

This example illustrated that the bone-like organ developed by the stem cell generator constructed from the active material described in Example 1 had a structure and function similar to that of autologous bone, and could repair autologous skull defects well, and was promising to be used in the repair of various bone defects.

### Example 5

Use of bone-like organs produced by stem cell generators in vivo for the treatment of autologous skull defects in old mice

The purpose of this example was to evaluate the therapeutic effect of the bone-like organ produced by the development of the stem cell generator manufactured in the same old mouse on the 5 mm diameter defect of the old mouse's skull.

The active material used was the scaffold containing rhBMP-2 described in Example 1.

The bone-like organs were produced by the development of stem cell generators in the animal body in Example 3.

Method:

SPF C57BL/6 mice, male, 52 weeks old, were randomly grouped. The experiment was grouped as follows.

| Group | Blank group | bone-like organ |
|---|---|---|
| Number | 6 | 6 |

Preparation of bone-like organ: the scaffold containing rhBMP-2 in Example 1 was subcutaneously implanted to produce bone-like organs after three weeks of development, and the bone-like organs were then removed and trimmed by using a punch with 5 mm inner diameter to obtain cylindrical bone-like organs with 5 mm diameter.

Autologous bone-like organ transplantation: After the mouse was anesthetized, the skin of the head of the mouse was cut open with a scalpel, and the skull was exposed. A circular saw with 5 mm outer diameter was used to create a 5 mm skull defect in the mouse, and the autologous bone-like organ prepared in the previous step was transplanted to the skull defect area. After the skin was sutured, the mouse was placed in a constant temperature stage to keep warm until the mice awoke. The samples were taken out for test at the established time point. The mice in the blank group only were made 5 mm skull defects, and then the wounds were sutured.

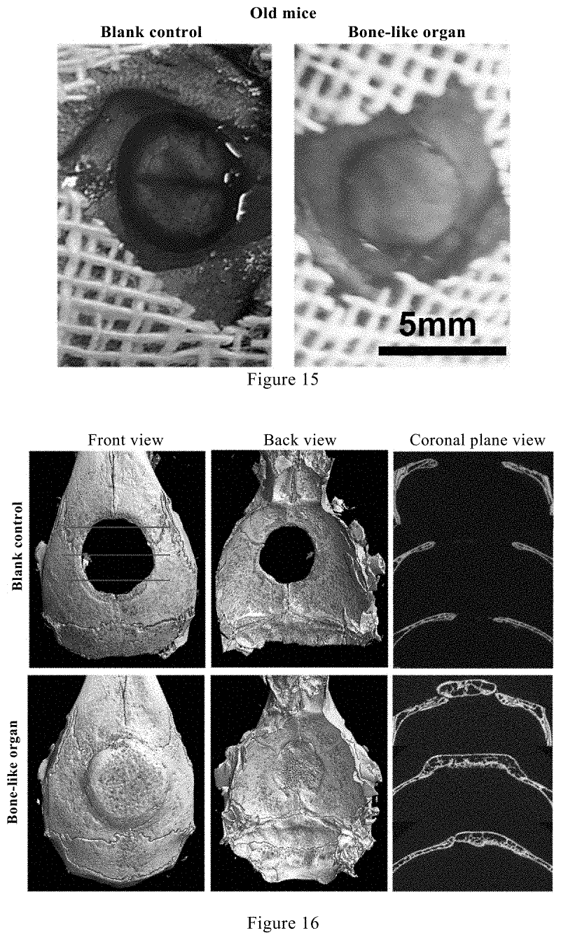

FIG. 15 showed an experimental process diagram of use of bone-like organs produced by the development of stem cell generators for treating autologous skull defects in old mice. The figure showed that after it was trimmed, the bone-like organ developed by the constructed stem cell generator in the body well covered the defect area, filled the bone defect part and strived to achieve the purpose of rapid repair.

FIG. 16 showed the μCT scan images of use of bone-like organs produced by the development of stem cell generators for treating autologous skull defects in old mice for 6 W. The figure showed that the bone-like organs produced by the development of stem cell generators quickly repaired bone defects.

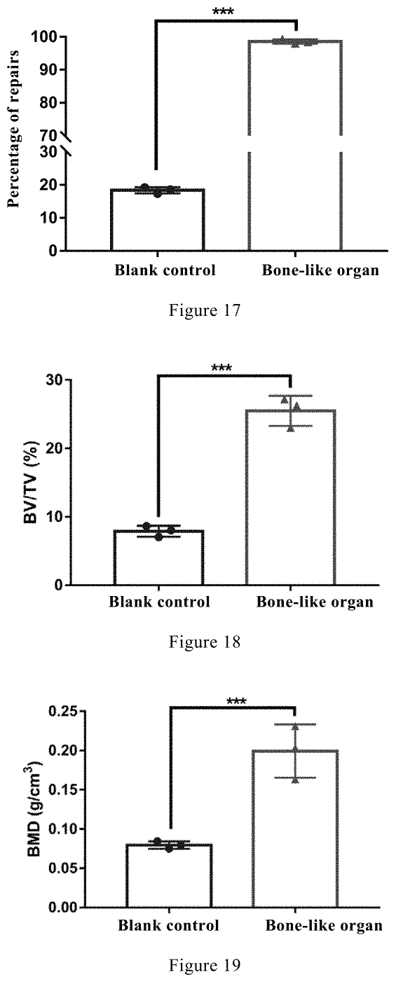

The quantitative data in FIG. 17 further showed that the bone-like organs produced by the development of stem cell generators achieved nearly 100% repair coverage of the bone defect.

FIGS. 18 and 19 showed that the BV/TV (bone volume/total volume) and BMD (bone mineralization density) of the repair site of bone-like organ produced by the development of stem cell generators were significantly higher than those of the blank control group, showing that bone-like organ produced by the development of stem cell generators had a better repair effect.

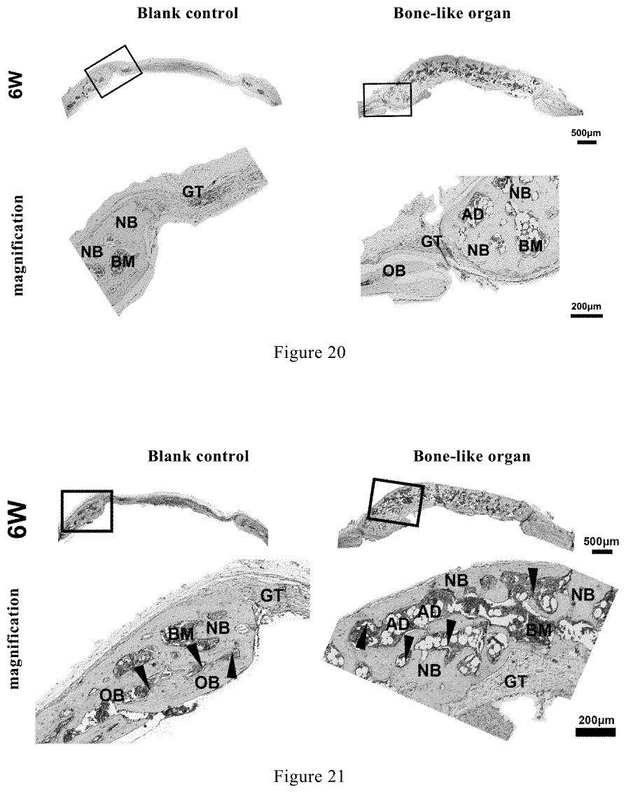

The H&E stained section images in FIG. 20 and the TRAP stained section images in FIG. 21 together indicated that the bone-like organ produced by the development of the stem cell generator could survive at the defect site after transplantation and effectively integrate with the defect edge to achieve a good repair effect.

This example illustrated that the stem cell generator constructed by the active material described in this example could be developed to have a structure and function similar to that of autologous bone, and could be used as a bone-like organ. It could also perform effective bone repair for elderly patients who are difficult to repair critical-sized bone defects. This method was expected to be applied to the repair of bone defects in various elderly patients with poor autologous bone condition.

All documents mentioned in the present invention are cited as references in this application, as if each document is individually cited as a reference. In addition, it should be understood that after reading the above teaching content of the present invention, those skilled in the art can make various changes or modifications to the present invention, and these equivalent forms also fall within the scope defined by the appended claims of the present application.

1. A stem cell generator, wherein the stem cell generator is formed by implanting a biomaterial with osteoinductive ability or a biomaterial loaded with an active substance and/or cell into an animal or human body to develop and generate an organoid, wherein the active substance is bone morphogenetic protein-2, bone morphogenetic protein-7, other growth factor/polypeptide having the ability to induce bone regeneration, growth factor/polypeptide combination, or a combination thereof; the cell is mesenchymal stem cell, and the mesenchymal stem cell is bone marrow-derived mesenchymal stem cell, adipose-derived mesenchymal stem cell, or mesenchymal stem cell from other sources; other type of cell having osteogenic differentiation ability; a cell assisting mesenchymal stem cell in osteogenic differentiation, such as vascular endothelial cell and the like.

2. The stem cell generator of claim 1, wherein the biomaterial is selected from one of collagen, gelatin, chitosan, alginic acid, hyaluronic acid, bacterial cellulose, polylactic acid, polyglycolide, polylactide, polyhydroxy fatty acid ester, polycarbonate, polycaprolactone, polyethylene glycol, polyfumaric acid, hydroxyapatite, calcium sulfate, tricalcium phosphate, tetracalcium phosphate, octacalcium phosphate, calcium metaphosphate, magnesium phosphate, pyrophosphate, calcium silicate, bioglass and decalcified bone matrix, or a copolymer/blend composition thereof.

3. The stem cell generator of claim 1, wherein the organoid contains pluripotent stem cell and bone marrow cell.

4. The stem cell generator of claim 1, wherein the pluripotent stem cell is hematopoietic stem/progenitor cell (HSC/HPC), mesenchymal stem cells (MSC) or other type of pluripotent stem cell.

5. The stem cell generator of claim 1, wherein the animal or human body refers to the muscle pocket, muscle space, intra-muscle, subcutis, or dorsal muscle of the abdominal cavity of the animal or human.

6. A method for preparing a bone graft/filler, comprising the following steps:
(1) implanting a biomaterial into an animal or human body;
(2) generating an organoid after development in the body to obtain the bone graft/filler, wherein,
the biomaterial is a biomaterial loaded with an active substance and/or cell, or a biomaterial having osteoinductive ability.

7. The method of claim 6, wherein the animal or human body refers to the muscle pocket, muscle space, intra-muscle, subcutis, or dorsal muscle of the abdominal cavity of the animal or human.

8. Use of the stem cell generator of claim 1 for the manufacture of a bone repair material.

9. The use of claim 8, wherein the bone repair material is used to treat spontaneous or trauma-induced bone defects or bone deformities.

10. The use of claim 8, wherein the bone repair material is used in the following occasions or disease treatment:
(1) for bone graft treatment of bone injury caused by trauma, bone nonunion, and delayed bone healing;
(2) for the treatment of spinal fusion and bone defect caused by bone tumor, osteoporosis, bone deformity and other diseases;
(3) for the treatment of bone defect in elderly patients with weak regenerative ability; or
(4) for the treatment of other diseases that require bone transplantation.
